(19) United States
(12) Patent Application Publication
(10) Pub. No.: US 2010/0297102 A1
(43) Pub. Date: Nov. 25, 2010

(54) SEROTONIN 5-HT3A RECEPTORS IN TREATMENT OF NEUROLOGICAL AND PSYCHIATRIC DISORDERS

(76) Inventors: Hannah Monyer, Heidelberg (DE); Dragos Inta, Heidelberg (DE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/305,819

(22) PCT Filed: Jun. 21, 2007
(86) PCT No.: PCT/IB2007/003556

§ 371 Date: Jul. 14, 2010

Related U.S. Application Data
(60) Provisional application No. 60/816,052, filed on Jun. 22, 2006.

(51) Int. Cl. A61K 39/395; G01N 33/00; A61K 31/497

(52) U.S. Cl. 424/130.1; 800/3; 514/253.06

(57) ABSTRACT

Methods are provided for modulating post-natal migration of neurons. Activation of the 5-HT3A receptor increases migration, while pharmacological blockade of the 5-HT3A receptor disrupts neuroblast migration into the cortex and hippocampus. These neuroblasts mature into GABAergic interneurons, and thus are relevant to conditions associated with disturbances in 5-HT levels and GABAergic inhibition.

The biogenic amine serotonin (5-hydroxytryptamine, 5-HT) is a brain neurotransmitter that has been strongly implicated in the pathophysiology and treatment of a wide variety of neuropsychiatric disorders. It signals through a myriad of pharmacologically defined cell surface receptors, only one of which (the 5-HT3 receptor) is a fast activating, ligand-gated, nonselective cation channel. Activation of 5-HT3 receptors produces a variety of effects including membrane depolarization and increase in intracellular Ca²⁺, modulation of neurotransmitter release, excitation of central and peripheral neurons, release of 5-HT from enterochromaffin cells of the small intestine, and Ca²⁺ influx into lymphocytes. 5-HT3 receptor activation mediates emetic and inflammatory responses and may contribute to pain reception, anxiety, cognition, cranial motor neuron activity, modulation of affect, and the behavioral consequences of drug abuse.

The 5-HT3A receptor subunit shares structural similarities with members of the superfamily of ligand-gated ion channels and is thought to be a pentameric protein with multiple agonist and allosteric ligand binding sites. Both native and recombinant 5-HT3 receptors reveal rapid and cooperative activation by agonists and desensitization to prolonged application of 5-HT. With a few exceptions, ligand-gated channels require the association of more than one kind of homologous subunit for function, and subunit composition determines the pharmacological and kinetic profile of heteromeric receptors, although the 5-HT3A subunit expressed in heterologous systems functions efficiently as homomers.

Neurogenesis occurs throughout life, particularly in the hippocampus, and the balance of neuronal loss and birth is essential in generating the plasticity necessary for new memory formation. In the adult mammalian hippocampus, production of new neurons is influenced by a variety of environmental and behavioral conditions. Factors that have been described as influencing neurogenesis, include, for example, exercise, diet, environmental stimulation, steroids, electroconvulsive therapy, and antidepressants.

The generation of new neurons is mediated by proliferating neural stem/progenitor cells, which are highly sensitive to local signaling. Stem cells represent the most immature cell necessary for neurogenesis. These cells give rise to more restricted precursors or progenitor cells and ultimately these progenitors differentiate into new functional neurons. These cells produce neurons in response to signals received from surrounding cells as well as humoral signals from circulating hormones, cytokines, and growth factors.

Classical birthdating studies using tritiated thymidine have led to the view that the generation of cortical neurons ceases before birth, except for limited sites of secondary neurogenesis in the SVZ, the dentate gyrus (DG) and the cerebellar external granular layer. The SVZ is the main site of adult neurogenesis. At perinatal stages, a dichotomy in the migratory pattern from the SVZ was postulated, with neuroblasts migrating anteriorly to the OB and glial cells migrating latero-dorsally.

Adult neurogenesis provides one mechanism of active structural plasticity in mature cortical circuits, which underlie learning and memory in the OB and in the hippocampus. Neuroblasts that are generated in the adult SVZ are thought to migrate only to the olfactory bulb, and hence neurogenesis in the SVZ has been thought to have little importance, if at all, for human brain functions and pathology. However, in humans, the majority of GABAergic interneurons arise from the prenatal ventricular zone and the SVZ. Also, newborn GABAergic interneurons have been found in deep cortical layers in adult rodents, thus indicating that the SVZ is an important source of neurogenesis in the rodent and human alike.

GABAergic interneurons can be classified into several subgroups, based on molecular, anatomical and functional criteria. There is increasing evidence that the region of origin and time of birth may also be critical determinants for interneuron diversity. GABAergic interneurons have been thought to be born during embryonic development in the subcortical ganglionic eminences (GE) and migrate tangentially into the cortex. Thus, they differ from excitatory pyramidal cells both with respect to their origin as well as their mode of migration.

Altered neuron migration and/or distribution in the brain may occur during mental illnesses such as depression, anxiety disorders, schizophrenia, and autism. For example, the hippocampus tends to be smaller in humans suffering from depression, anxiety disorders such as PTSD, and schizophrenia. Furthermore, many clinically efficacious anti-depressant medications enhance neurogenesis in the hippocampus.

There is a need to develop compounds and methods of treatment that prevent the onset, and/or ameliorate the symptoms, of diseases associated with damage, dysfunction,or degeneration of neurons. There is also a need to develop compounds and methods of treatment to improve the regeneration and repair of neurons in the brain, and for methods of screening compounds active in these areas.

### SUMMARY OF THE INVENTION

Methods are provided for modulating post-natal migration of neurons through interaction with 5-HT3A receptors present in migratory neuroblasts. Activation of the 5-HT3A receptor enhances migration, while pharmacological blockade of the 5-HT3A receptor disrupts neuroblast migration into the cortex and hippocampus. The migratory neuroblasts may be characterized by expression of doublecortin, Tuj1 and PSA-NCAM. These neuroblasts mature into GABAergic interneurons, and thus are relevant to conditions associated with disturbances in 5-HT levels and GABAergic inhibition.

In one embodiment of the invention, methods are provided for treating a subject having or being susceptible to a neurological disorder associated with impaired 5-HT3 receptor signaling, e.g. neuropsychiatric disorders such as autism, schizophrenia, major depressive disorders, obsessive-compulsive disorders, anxiety disorders, drug addiction, cerebral ischemia and epilepsies, plasticity of neuronal circuits in the postnatal brain, and the like. The method entails administering to the subject a prophylactic or therapeutically effective amount of an agent that modulates migration of postnatal to adult 5-HT_{3A} receptor positive neuroblasts.

In another embodiment of the invention, methods are provided for screening drug candidates for effectiveness in modulating the migration of postnatal to adult 5-HT3A receptor positive neuroblasts. Screening assays may utilize the transgenic animal models as described herein, for example where the migratory neuroblasts are marked with a fluorescent protein that allows tracking of migration. Candidate compounds are administered to a postnatal animal, and the effect of the compound on neuroblast migration is determined. Alternative screening methods include selection of candidate agents based on receptor binding, binding to neuroblasts in culture, etc, which screening is optionally followed with in vivo assays. Screening may be used to identify agents that selectively target specific cells to improve targeting specificity of the intervention.

The examples provided herein demonstrate a pancortical pattern of migration of specific GABAergic interneurons from the subventricular zone in the postnatal brain and the regulation of this migration by serotonin 5-HT3 receptors. The subventricular zone is therefore shown to be a reservoir of newly generated inhibitory GABAergic neurons for many other brain regions in the postnatal brain, including the cortex, olfactory bulb, basolateral amygdala, striatum and nucleus accumbens. Blockade of 5-HT3A receptors results in disruption of migration of neuroblasts in cortex and hippocampus in the early postnatal brain.

### BRIEF DESCRIPTION OF THE DRAWINGS

FIG. 1. Generation of transgenic mice and specificity of EGFP expression. (A) Representation of the native and modified 5-HT_{3A} gene structure, containing the EGFP coding sequence, located on a BAC. (B) Application of 100 μM 5-HT to whole-cell voltage clamped EGFP-positive neurons evoke a 5-HT₃ receptor-mediated inward current that can be blocked by the specific antagonist MDL72222 (n=5) (C) All 39 randomly chosen EGFP-positive neurons in different brain areas had functional 5-HT3 receptor-mediated inward currents. Bars represent the average peak amplitude. The number of recorded cells is displayed in the bars. (D to G) Specific EGFP (green) staining in the hippocampus (D), cortex (E), BLA (F) and OB (G) of adult transgenic mice. Cx—cortex; so—stratum oriens; sp—stratum pyramidale; sr—stratum radiatum; slm—stratum lacunosum-moleculare. Scale bar, 200 μm.

FIG. 2. Cells with migratory morphology in the streams and lower cortical layers are postmitotic and mitotic neuroblasts. (A) EGFP immunocytochemistry (red) in the RMS and DMS in sagittal sections from P10 transgenic animals and in cells detached from the RMS (enlargement of the boxed area). (B to E) Co-localization of EGFP (green) and DCX (red) double staining in the RMS (B) and in cells detached from the RMS that are directed towards the cortex (C; enlargement of the boxed area in B) in sagittal sections at P9 and in the EMS (D and E) in horizontal sections at P16. Several DCX/EGFP-double positive cells with leading processes oriented radially towards the cortical surface (arrows), as well as double labeled cells with a more ‘mature’ morphology (asterisc) are visible in lower cortical layers (D) (E, enlargement of boxed area in D). (F) Widely branched cell in layer V of the parietal cortex of wild-type mice at P14, co-expressing DCX (red) and NeuN (green), indicative of an intermediate stage between an immature and a mature neuron. In triple-labelings, similar cells showed co-localizations of all 8 three markers (EGFP, NeuN, DCX). (G to I) Co-localization of EGFP (green) and BrdU (red) after BrdU injections. (G) Co-localization of EGFP (green) and BrdU (red) at P25 after BrdU Injections at E14.5. (H) Co-localization of EGFP (green) and BrdU (red) subsequent to BrdU injection at P7 in neuroblasts of the DMS at P16, hence 9 days post injection (H) and in cortical layer IV at P32, hence 25 days post injection (I). Similar results were obtained in triple-labeling experiments (EGFP, NeuN, DCX). CC—corpus callosum; Cg—cingulate cortex; CPu—caudate putamen; Cx—cortex; Hi—hippocampus. Scale bars in (A) 50 μm, (B) 150 μm, (C) 50 μm, (D) 120 μm, (E) 75 μm, (F) 25 μm, (G, H, I) 10 μm.

FIG. 3. Neuroblasts in the streams express functional 5-HT3 receptors and migrate from the streams into the cortex. (A) Schematic representation of the location of neuroblasts recorded from the DMS. (B) Two categories of neuroblasts: immature neuroblasts displayed no action potentials (red dots), whereas more mature neuroblasts responded to current injection with one or more action potentials, displaying a fast voltage-dependent inward Na. current (green dots). (C) In the DMS, 50 cells were recorded: 37 were mature/spiking, 13 were immature/non-spiking. 18 out of 22 mature neuroblasts responded with a 5-HT3 receptor-mediated inward current upon application of 5-HT (average amplitude: −46.0±10.5 pA). In contrast; only in 2 of 13 immature neuroblasts, a 5-HT3 receptor-mediated inward current was detected, with an amplitude of −15 and −20 pA. (D) Migration of neuroblasts from the RMS into the cortex. For the cell indicated with an arrow the speed of migration was 9 μm/h. Asterisc indicates another migrating neuroblast. The size of each frame is 60×240 μm.

FIG. 4. Effect of 5-HT3 antagonists on neuroblast migration from the SVZ. (A and B) Matched control and sagittal organotypic slices from P3 transgenic mice treated with tropisetron for 6 9 days. Confocal image of EGFP staining showing the decrease of the number of EGFP-labeled cells (green) in deep cortical layers and stratum radiatum of the hippocampus, concomitant with an increase in cell density in the DMS, in slices treated with tropisetron (A) compared to control (B). FIG. 4C shows a graphical representation. Error bars represent s.e.m. Asteriscs denote a significant difference (*p<0.001) assessed by unpaired Student's t-test. (D) Models illustrating the migratory pattern of EGFP-positive cells from the early postnatal SVZ. The three migratory streams (green) and the directions of cell migration (green arrows) are shown in sagittal and horizontal brain sections. Cx—cortex; DG—dentate gyrus; Hi—hippocampus; sr—stratum radiatum; slm—stratum lacunosum-moleculare. Scale bars, (A and B) 200 □m.

FIG. 5. Specificity of EGFP expression. (A and B) Radioactive in situ hybridization experiments show similar mRNA expression pattern of EGFP and 5-HT_{3A} receptors in brain coronal (A) and 13 sagittal (B) sections from adult transgenic mice. Note the signal of scattered cells in the cortex, the specific labeling in stratum lacunosum-moleculare, the subgranular layer of the DG of the hippocampus and BLA as well as the lack of mRNA expression in the thalamus (A) and the high signal in the RMS (B). The exposure time for the sections shown in (A) was longer than for those in (B), accounting for the difference in the intensity of the signal in the cortex.

FIG. 6. Co-localization of EGFP with markers for mature neurons and lack of co-localization with markers for mature glial cells and oligodendrocytes. (A) Co-localization of EGFP (green) and NeuN (red) in stratum radiatum of the adult hippocampus. (B) No co-localization of EGFP (green) with GFAP (red) in the SVZ in P10 animals. Similar results were obtained for all regions of the cortex in young and adult animals. (C) No co-localization of EGFP (green) and CNP (red) in the cortex at P16 (shown is layer III). Similar results were obtained also in adult animals. sp—stratum pyramidale; sr—stratum radiatum. Scale bars. 50 μm in (A and C) and 20 μm in (B).

FIG. 7. Co-localization of EGFP with interneuron-specific markers at P10. (A to F) Doublelabeling showing co-localization in cortex (layer II-III) of EGFP (green) and GABA (red) (A to C) and CR (D) but not with parvalbumin (E) and somatostatin (hippocampus) (F). Similar results were obtained in the adult. Scale bars, 50 μm.

FIG. 8(A) EGFP (red) immunohistochemistry in the EMS visible on horizontal sections from P10 transgenic mice. (B) In horizontal sections at more superior levels, many EGFP-positive chains of cells (red) oriented along structures of the callosal system can be visualized at P10 being. Cx—cortex; Cg—cingulate cortex; Par—parietal cortex; CPu—caudate putamen; LV—lateral ventricle; Hi—hippocampus. Scale bars, 200 μm.

FIG. 9. Migration from the SVZ into cortical layers continuous in the adult brain. (A and B) Numerous EGFP-positive cells (red) with the specific morphology of neuroblasts (arrows) located outside of the RMS in the corpus callosum and directed towards layer IV, as visible in sagittal sections from adult transgenic mice. Cx—cortex; CPu—caudate putamen. Scale bars, 200 μm in (A) and 100 μm in (B).

FIG. 10. BrdU labeling (red) after injections at E12.5 (A), E14.5 (B), E16.5 (C) and P7 (D) in sagittal sections of the frontal cortex at P25. (E to G) Reduction of EGFP-positive cell number at P16 in the granule cell layer and periglomerular cell layer of the OB in mice injected at P7 with 50 mg/kg BrdU(F) and 300 mg/kg BrdU (G) compared to untreated transgenic control mice (E). Scale bars, 200 μm in (A to D), 100 μm in (E to F).

### DETAILED DESCRIPTION OF THE EMBODIMENTS

Methods are provided for modulating post-natal migration of neurons through interaction with 5-HT3A receptors present in migratory neuroblasts. In one embodiment of the invention, a subject having or being susceptible to a neurological disorder associated with impaired 5-HT3 receptor signaling, e.g. neuropsychiatric disorders such as autism, schizophrenia, major depressive disorders, obsessive-compulsive disorders, anxiety disorders, drug addiction, cerebral ischemia and epilepsies, plasticity of neuronal circuits in the postnatal brain, and the like, is treated by modulation of post-natal migration of neuroblasts. The method entails administering to the subject a prophylactic or therapeutically effective amount of an agent that modulates migration of postnatal to adult 5-HT3A receptor positive neuroblasts. In another embodiment of the invention, methods are provided for screening drug candidates for effectiveness in modulating the migration of postnatal to adult 5-HT3A receptor positive neuroblasts.

Screening assays may feature the use non-human transgenic animal models. Transgenic animals of interest comprise a detectable marker operably linked to a promoter selectively active in post-natal migratory neuroblasts, e.g. 5-HT3A promoter, doublecortin promoter, Tuj1 promoter, PSA-NCAM promoter, etc. Screening assays may include control assays utilizing known 5HT3A agonists or agonists for a comparison with the activity of a candidate agent. Screening for selectivity of agents is also of interest, e.g. comparing the activity of an agent in activating or inhibiting one or more of 5-HT1A; 5-HT1B; 5-HT1D; 5-HT1E; 5-HT1 F; 5HT2A; 5-HT2B; 5-HT2C; 5-HT3A; 5-HT3B; 5-HT4; 5-HT5A; 5-HT5B; 5-HT6; 5-HT7; etc., where preferred agents selectively activate or inhibit 5-HT3A. The screening methods are of particular use for determining the specificity and action of antidepressant or antipsychotic drugs.

Such animals are useful for testing the specificity of drugs developed as 5-HT3A receptor-selective agonists and antagonists. Completely selective compounds will not interact with other receptors, and thus will act only on migration of neuroblasts as described herein. The animals are also useful in determining to determine the extent to which such migratory neuroblasts contribute to the neurological disorders and treatment thereof.

The term neurogenesis refers to the generation of new neurons from proliferating neural stem/progenitor cells. Neural stem cells give rise to more restricted precursors or progenitor cells and ultimately these progenitors differentiate into new functional neurons. The term “neural stem cells” (NSCs) includes “neural progenitor cell,” “neuronal progenitor cell,” “neural precursor cell,” and “neuronal precursor cell” (all referred to herein as NPCs).

Neural stem and progenitor cells can participate in aspects of normal development, including migration along migratory pathways to disseminated CNS regions, differentiation into multiple developmentally- and regionally-appropriate cell types in response to microenvironmental cues, and non-disruptive, non-tumorigenic interspersion with host progenitors and their progeny. As such, these cells find use in the treatment of a variety of conditions, including traumatic injury to the spinal cord, brain, and peripheral nervous system; treatment of degenerative disorders including Alzheimer's disease; Huntington's disease; Parkinson's disease; affective disorders including major depression; stroke; and the like.

In vivo, neural stem cells (NSCs) are located within the ependymal and/or subventricular zone (SVZ) lining the lateral ventricle and in the dentate gyrus of the hippocampus formation. Asymmetric division of NSC maintains their number, while generating a population of rapidly dividing precursor or progenitor cells. The progenitors respond to a range of cues that dictate the extent of their proliferation and their fate, both in terms of the cell type that they differentiate into and the position that they ultimately take up in the brain.

By “modulating neuroblast migration” as used herein, it is meant that the movement of post-natal neuroblasts away from the subventricular zone in the brain is altered relative to the movement in the absence of a treatment. For example, inhibition of 5-HT3A signaling will reduce the neuroblast migration, while activating 5HT3A will increase movement. The net effect is to alter the distribution of GABAergic interneurons in sites distant from the SVZ. Generally such an increase or decrease is relative, as compared to a comparable situation in the absence of the treatment.

As used herein, an “agonist” is a molecule which, when interacting with, (for example binding to) a target protein such as the 5-HT3A receptor, increases or prolongs the amount or duration of the effect of the biological activity of the target protein. Agonists are preferably selective for the target protein, e.g. by activating the 5HT3A receptor but not other serotonin receptors. Agonists of these molecules are known in the art, including the relatively non-selective agonists 5-hydroxytryptamine, 5-HT3 selective agonists include quipazine; thiazole derivatives that behave as selective agonists of the 5-HT3 serotonin receptor (U.S. Pat. No. 5,656,637); 2-methyl-5-hydroxytryptamine, m-chlorophenylbiguanide (m-CPBG); etc.

By contrast, the term “antagonist,” as used herein, refers to a molecule which, when interacting with, (for example binding to) a target protein such as the 5-HT3A receptor decreases the amount or the duration of the effect of the biological activity. Antagonists of these molecules are known in the art. For example, the 5-HT3A receptor antagonists include renzapride; S-zacopride; tropisetron; Aloxi (palonosetron); dolasetron (anzemet®); granisetron (kytril®); ondansetron (zofran®)

Agonists and antagonists may include proteins, nucleic acids, carbohydrates, antibodies, or any other molecules that decrease the effect of a protein. Unless otherwise specified, the term “agonist” can be used interchangeably with “activator”, and the term “antagonist” can be used interchangeably with “inhibitor”.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

In one aspect of the invention, candidate agents are screened for the ability to modulate neuroblast migration. Such compound screening may be performed using an in vitro model, a genetically altered cell or animal, or purified protein. A wide variety of assays may be used for this purpose. In one embodiment, compounds are initially identified as being active in binding assays with the 5-HT3A receptors, or are predicted to be antagonists or agonists of the receptors, or activators or inhibitors of specific downstream signaling molecules, transcription factors, or genes, are then tested for in vivo effectiveness in modulating neuroblast migration.

For example, candidate agents may be identified by known pharmacology, by structure analysis, by rational drug design using computer based modeling, by binding assays, and the like for interaction with 5HT3A, preferably selective interaction with 5HT3a relative to other serotonin receptors. Such candidate compounds may then be used to contact neuroblasts in an in vitro or in vivo environment to determine effectiveness in modulating neuroblast migration.

The term “agent” as used herein describes any molecule, e.g. protein or pharmaceutical, with the capability of modulating neurogenesis by acting through excitation pathways of neural progenitor cells. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally a plurality of assay mixtures are run in parallel with different agent concentrations to obtain a differential response to the various concentrations. Typically one of these concentrations serves as a negative control, i.e. at zero concentration or below the level of detection.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt at al., Proc. Nat. Acad. Sci. USA 90:6909-13, 1993; International Patent Publication WO 94/08051; Baum, Chem. & Eng. News, 72:20-25, 1994; Burbaum at al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Compounds that are initially identified by any screening methods can be further tested to validate the apparent activity. The basic format of such methods involves administering a lead compound identified during an initial screen to an animal that serves as a model for humans and then determining the effects on neuroblast migration. The animal models utilized in validation studies generally are mammals. Specific examples of suitable animals include, but are not limited to, primates, mice, and rats.

In some embodiments, transgenic animals are used in screening assays. The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, preferably a permanent genetic change, is induced in a cell following incorporation of exogenous DNA. A permanent genetic change is generally achieved by introduction of the DNA into the genome of the cell. Vectors for stable integration include plasmids, retroviruses and other animal viruses, YACs, and the like. Of interest are transgenic mammals, e.g. cows, pigs, goats, horses, etc., and particularly rodents, e.g. rats, mice, etc.

Transgenic animals comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

Transgenic animal of particular interest for the methods of the invention comprise a detectable marker operably linked to a promoter active in post-natal migratory neuroblasts. Examples of such promoters include, without limitation, the 5HT3A promoter, doublecortin promoter, PSA-NCAM promoter; etc. Such promoters are known in the art and publicly available.

For example, serotonin (5-hydroxytryptamine; 5-HT) is a neurotransmitter in the central and peripheral nervous systems that plays a role in many physiologic processes such as sleep, appetite, thermoregulation, pain perception, hormone secretion, and sexual behavior. Abnormality of the serotonergic system has been implicated in a number of human disorders such as mental depression, migraine, epilepsy, obsessive-compulsive disorder, and affective disorder. Like other neurotransmitters, 5-HT is released into the synaptic junction and exerts its effect on specific receptors on the postsynaptic membranes. Based on differential radioligand binding affinities, several 5-HT receptors have been identified. Maricq et al. (1991) cloned the mouse 5-hydroxytryptamine receptor 3A (Htr3) gene. Miyake et al. (1995) isolated human HTR3A cDNA from a hippocampus cDNA library. This receptor is a ligand-gated ion channel, whereas all other known 5-HT receptor subtypes are G protein-coupled receptors. The deduced HTR3A protein contains 478 amino acids with a potential signal peptide of 23 residues. The single-channel conductance for homomers of HTR3A is 0.4 pS. Sequence information for 5-HT3A gene and promoter is known and publicly available, e.g. see Bruss at al. (2000) Neuropharmacology 39:308-315; Maricq et al. (1991) Science 254: 432-437, each herein specifically incorporated by reference.

Other promoter sequences of interest include doublecortin and its promoter. Doublecortin is associated with migration of cortical neurons. The gene contains 9 exons and encodes a 40-kD predicted protein of 360 amino acids. The gene appears to be expressed exclusively in fetal brain as an mRNA species of approximately 9.5 kb. The product is apparently required for neuronal migration. Sequence information for doublecortin and promoter is known and publicly available, e.g. see Karl et al. (2005) J. Neurochem. 92:264-282.

Neuron specific class III beta-tubulin is also expressed by post-natal migratory neuroblasts, see, for example, Menenzes and Luskin (1994) J. Neuroscience 14:5399-5416; and Sullivan (1988) Ann. Rev. Cell Bio. 4:687-716.

The polysialic acid-neural cell adhesion molecule (PSA-NCAM) is also expressed on neurons, including post-natal migratory neuroblasts during development, see Bruses and Rutishauser (1998) J. Cell. Biol. 140:1177-1186.

In a preferred embodiment of the invention, the detectable marker operably linked to a promoter active in post-natal migratory neuroblasts is a fluorescent or luminescent protein, e.g. GFP, firefly luciferase, etc., as is known in the art. By “operably linked” is meant that a DNA sequence and a regulatory sequence(s) are connected in such a way as to permit gene expression when the appropriate molecules, e.g. transcriptional activator proteins, are bound to the regulatory sequence(s).

The native gene encoding green fluorescent protein has been cloned from the bioluminescent jellyfish Aequorea victoria (Morin, J. et al., J Cell Physiol (1972) 77:313-318). The availability of the gene has made it possible to use GFP as a marker for gene expression. GFP itself is a 283 amino acid protein with a molecular weight of 27 kD. It requires no additional proteins from its native source nor does it require substrates or cofactors available only in its native source in order to fluoresce. (Prasher, D. C. et al., Gene (1992) 111:229-233; Yang, F. et al., Nature Biotechnol (1996) 14:1252-1256; Cody, C. W. et al., Biochemistry (1993) 32:1212-1218.) Mutants of the GFP gene have been found useful to enhance expression and to modify excitation and fluorescence. GFP-S65T (wherein serine at 65 is replaced with threonine) may be used, which has a single excitation peak at 490 nm. (Heim, R. et al., Nature (1995) 373:663-664); U.S. Pat. No. 5,625,048. Other mutants have also been disclosed by Delagrade, S. et al., Biotechnology (1995) 13:151-154; Cormack, B. at al., Gene (1996) 173:33-38 and Cramer, A. at al. Nature Biotechnol (1996) 14:315-319. Additional mutants are also disclosed in U.S. Pat. No. 5,625,048. By suitable modification, the spectrum of light emitted by the GFP can be altered. Thus, although the term “GFP” is used in the present application, the proteins included within this definition are not necessarily green in appearance. Various forms of GFP exhibit colors other than green and these, too, are included within the definition of “GFP” and are useful in the methods and materials of the invention. In addition, it is noted that green fluorescent proteins falling within the definition of “GFP” herein have been isolated from other organisms, such as the sea pansy, Renilla renformis. Any suitable and convenient form of the GFP gene can be used in the methods of the invention.

Animals, including transgenic animals described herein are useful in evaluating the selectivity and efficacy of candidate compounds that modulate migration of post-natal neuroblasts. For example, sections of brain tissue may be evaluated for the presence of migratory neuroblasts in response to agents that interact with 5HT3A, where agonists increase migration and antagonists block migration. Sections of interest for tracking include the lower cortical layers, SVC, hippocampus, corpus callosum, cingulate cortex, caudate putamen, cortex, etc. The detectable marker provides a means of readily tracking the neuroblasts, although traditional methods of immunocytochemistry provide an alternative means of tracking cells, for example where antibodies specific for 5HT3A are used to stain sections. In another embodiment, cells in live animals can be tracked with fluorescent detectable markers (see Contag et al. (2000) NeoReviews, Vol. 1:e -232, herein specifically incorporated by reference for teachings of tracking methods).

In some embodiments, known modulators of 5HT3A function may be used as positive or negative controls for testing a candidate agent. As shown in the examples, 4-HT3 antagonists decrease the number of migratory neuroblasts in deep cortical layers and stratum radiatum of the hippocampus.

The methods of the invention may be used to enhance neuroblast migration in patients suffering from neurodegenerative disorders. In many instances the individual will be human, however treatment of other mammals is also contemplated, for research purposes, e.g. mice, rats, etc., and/or for veterinary purposes, e.g. horses, dogs, cats, cows, monkeys, etc.

Patients suffer neurological and functional deficits after stroke, CNS injury, and neuropsychiatric/neurodegenerative disease. These findings provide a means to enhance brain compensatory mechanisms to improve function after CNS damage or degeneration. The induction of migration will promote functional improvement after stroke, injury, aging, neuropsychiatric and neurodegenerative disease. This approach can also provide benefit to patients suffering from other neurological diseases including Alzheimer's disease, Parkinson's disease, Huntington's disease, depression, schizophrenia, PTSD, autism, bipolar disorder, etc. Diseases in which defects in neurogenesis are implicated also include Lewy Body dementia, Frontotemporal dementia/Pick's disease, AIDS dementia complex, dementia puligistica and chronic cognitive dysfunction following head trauma, prion-associated dementia such as Creutzfeldt-Jacob disease, cognitive dysfunction following chronic seizure disorders or an episode of, status epilepticus, cognitive dysfunction following encephalitis or meningitis, amyotrophic lateral sclerosis (ALS)/parkinsonian/dementia complex of Guam.

Autism, often referred to as autistic disorder or infantile autism, is a complex behavioral disorder which, by definition, develops prior to age three years. Autism is defined completely on the basis of impairments in social interaction, impairments in communication, and repetitive and stereotypic behaviors. For most children, the onset of autism is gradual; however, approximately 30% have a “regressive” onset. Fifty to seventy percent of children with autism are defined as mentally retarded by nonverbal IQ testing. Seizures develop in approximately 25% of children with autism.

The standard diagnostic criteria for autism, compiled by the American Psychiatric Association Manual of Psychiatric Diseases, 4th edition (DSM-IV), are the primary diagnostic reference used in the United States. The standard diagnostic criteria include qualitative impairment in social interaction, as manifested by at least two of the following; qualitative impairments in communication; stereotyped and repetitive use of language or idiosyncratic language; lack of varied, spontaneous make-believe play or social imitative play appropriate to developmental level; restricted repetitive and stereotyped patterns of behavior, interests, and activities. Criteria also include delays or abnormal functioning in at least one of the following areas, with onset prior to age three years: social interaction, language as used in social communication, or symbolic or imaginative play.

Clinical depression manifests as major depressive disorder, with episodic course and varying degrees of residual manifestations between episodes. The diagnosis of clinical depression is usually straightforward, but recognizing low-grade symptoms may be difficult. For example, in major depressive disorder with incomplete recovery, classic depressive symptoms recede and are replaced by subacute or chronic hypochondriacal concerns, irritable morosity, and secondary interpersonal trouble in conjugal life. In other patients, considered masked depressives, depression may not be consciously experienced. Others complain of fatigue, various aches and pains, fears of calamity, and fears of becoming insane. REM latency is shortened in these patients, supporting the affective nature of the clinical presentations. Psychotic manifestations, which occur in 15% of melancholic patients, are a hallmark of a delusional or psychotic depressive subtype. In atypical depression, reverse vegetative features dominate the clinical presentation; they include anxious-phobic symptoms, evening worsening, initial insomnia, hypersomnia that often extends into the day, and hyperphagia with weight gain. Unlike patients with melancholia, those with atypical depression show mood brightening to potentially positive events but often crash into a paralyzing depression with the slightest adversity. Atypical depressive and bipolar II disorders overlap considerably.

Obsessive-compulsive disorder is characterized by recurrent, unwanted, intrusive ideas, images, or impulses that seem silly, weird, nasty, or horrible (obsessions) and by urges to do something that will lessen the discomfort due to the obsessions (compulsions). Obsessive-compulsive disorder occurs about equally in men and women and affects 1.6% of the population during any 6-mo period. The overarching obsessional theme is harm, risk, or danger, and common obsessions include contamination, doubt, loss, and aggressivity. Typically, persons with obsessive-compulsive disorder feel compelled to perform repetitive, purposeful, intentional behaviors called rituals to balance their obsessions. Most persons with obsessive-compulsive disorder are aware that their obsessions do not reflect real risks and that the physical and mental behaviors they perform to relieve their concern are unrealistic and excessive to the point of being bizarre. Preservation of insight, although sometimes slight, differentiates obsessive-compulsive disorder from psychotic disorders, in which contact with reality is lost. Because persons with this disorder fear embarrassment or stigmatization, they often conceal their obsessions and rituals, on which they may spend several hours each day. Depression is a common secondary feature, present in about ⅓ of patients at the time of diagnosis and in ⅔ at some point in their lifetime.

By “neurological” or “cognitive” function it is meant that the neuroblast migration in the brain enhances the patient's ability to think, function, etc. Humans treated by the methods of the invention have increased production of brain cells that facilitate improved cognitive, memory, and motor function as well as improved mood, thought process, or anxiety regulation.

In one embodiment, the region of increased neuroblast migration is within the hippocampus. In another embodiment, the region of increased neurogenesis is within the cortex, striatum, substantia nigra, or other brain region. In another embodiment, a region of increased neurogenesis results from migration of newly divided neurons within the brain, or transplantation of NPC, which may be autologous or allogeneic. The newly divided neurons may migrate to the corpus callosum, striatum, cortex, septum, basal ganglion, nucleus basalis, etc. The amount of increased neuroblast migration may comprise at least a measurable increase relative to a control lacking such treatment, for example at least a 10% increase, at least a 20% increase, at least a 50% increase, or more.

The effective amount of a therapeutic composition to be given to a particular patient will depend on a variety of factors, several of which will be different from patient to patient. Utilizing ordinary skill, the competent clinician will be able to optimize the dosage of a particular therapeutic or imaging composition in the course of routine clinical trials.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as impairment in memory or learning ability or other cognitive function. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

As mentioned above, in these applications an effective amount of a 5HT3A modulating agent, i.e. agonist or antagonist, is administered to the host. By “effective amount” is meant a dosage sufficient to produce a desired result, where the desired result is generally an amelioration or alleviation, if not complete cessation, of one or more symptoms of the disease being treated, particularly the cognitive impairment symptoms, e.g., memory, learning ability, and the like.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In addition to the above methods of treatment, the subject methods also find use in the prophylactic or preventative treatment regimens. In such methods, the host is administered an amount of a 5HT3A modulating agent, typically according to a dosage schedule (.e.g., daily, weekly, monthly etc.), that is sufficient to prevent the occurrence of at least symptoms of the disorder.

Administration of the treatment is maintained for a period of time sufficient to effect a change in the targeted function. Such treatment may involve dosing for at least about one week, at least about two weeks; at least about 3 weeks; at least about one month; at least about two months; at least about four to six months; or longer, for example at least about one or more years. For extended treatment; e.g. treatment of one or more years, a schedule may involve intermittent periods, such as one week on and one week off; two weeks on and two weeks off; one week in a month, etc.

Patients that can benefit from the present invention may be of any age and include adults and children, e.g. young adults. Children, e.g. neonate, infant, early childhood, adolescent, etc. in particular may benefit prophylactic treatment. Children suitable for prophylaxis can be identified by genetic testing for predisposition, e.g. by chromosome typing; by family history, or by other medical means. As is known in the art, dosages may be adjusted for pediatric use.

The 5HT3A modulating agent is generally administered to the host as a pharmaceutical composition that includes an effective amount of the 5HT3A modulating agent in a pharmaceutically acceptable vehicle. In the subject methods, the active agent(s) may be administered to the host using any convenient means capable of resulting in the desired improvement on cognitive function.

Therapeutic agents can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

Many 5HT3A modulating agents are known to be bioactive in the central nervous system after oral or parenteral administration. For those that are not, one strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant for example.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

Toxicity and therapeutic efficacy of the active ingredient can be determined according to standard pharmaceutical procedures in cell cultures and/or experimental animals, including, for example, determining the LD₅₀ (the dose lethal to 50% of the population, or for the methods of the invention, may alternatively by the kindling dose) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NE) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g. intravascular (intravenous, intraarterial, intracapillary) administration, injection into the cerebrospinal fluid, intracavity or direct injection in the brain. Intrathecal administration maybe carried out through the use of an Ommaya reservoir, in accordance with known techniques. (F. Balis et al., Am J. Pediatr. Hematol. Oncol. 11, 74, 76 (1989).

The effective amount of a therapeutic composition to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

The following examples are offered by way of illustration and not by way of limitation.

### Experimental

A high level of 5-HT has been shown to be beneficial in several mood disorders, but the mechanism of action of drugs of the selective-serotonin reuptake inhibitors (SSRI's) class or the class of 5-HT receptors that mediate this action are not known. Treatment with SSRI's becomes effective only after 3-4 weeks of treatment, suggesting the occurrence of long-lasting plastic changes in neuronal circuits and neurogenesis induced by 5-HT, and not only immediate receptor-mediated effects. 5-HT3A receptors are the first serotonin (5-HT) receptors demonstrated to be expressed both by neuroblasts and Cajal-Retzius cells, therefore 5-HT3A receptors are likely to mediated at least some of the effects of 5-HT in neurogenesis. Postnatal deficits in the level of brain 5-HT were demonstrated in autistic childrens, (Chugani et al, 1999) as well as alterations of the inhibitory GABAergic component of cortical columns (Casanova et al., 2003). Disturbances in several regions adjacent to the subventricular zone or the rostral migratory stream in rodents were associated with neuropsychiatric disorders in humans.

GABAergic interneurons are born during embryonic development in the ganglionic eminences and migrate tangentially into the cortical plate. GABAergic interneurones that are born postnatally and in the adult are thought to populate ‘only’ the OB (olfactory bulb) and their site of origin is the SVZ (subventricular zone).

The initial goal of this study was to identify and functionally characterize 5-HT3 receptor expressing neurons in the hippocampus and cortex. This required ‘marking’ of a scarce cell population since, although 5-HT₃ receptors are widely distributed in the central and peripheral nervous system, their expression in the hippocampus and cortex is restricted to a subpopulation of GABAergic interneurons. These interneurons receive serotonergic Input from the raphe nuclei, and it is not clear which function they subserve in the brain. The 5-HT₃ receptors are the only ionotropic receptors in the serotonin receptor family and of the two subunits 5-HT_{3A} and 5-HT_{3B}, only the former is expressed in the brain.

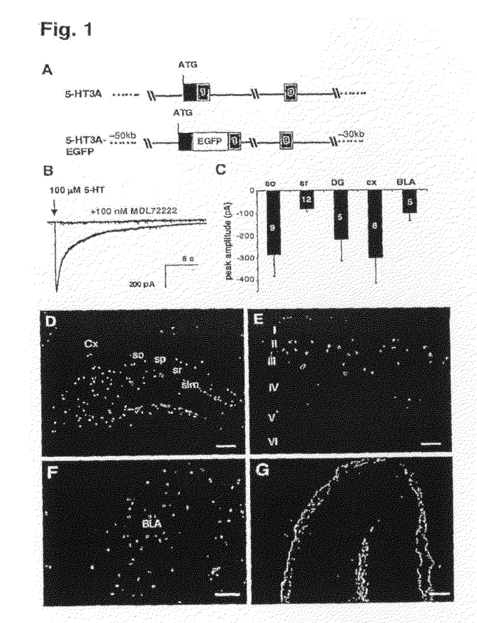

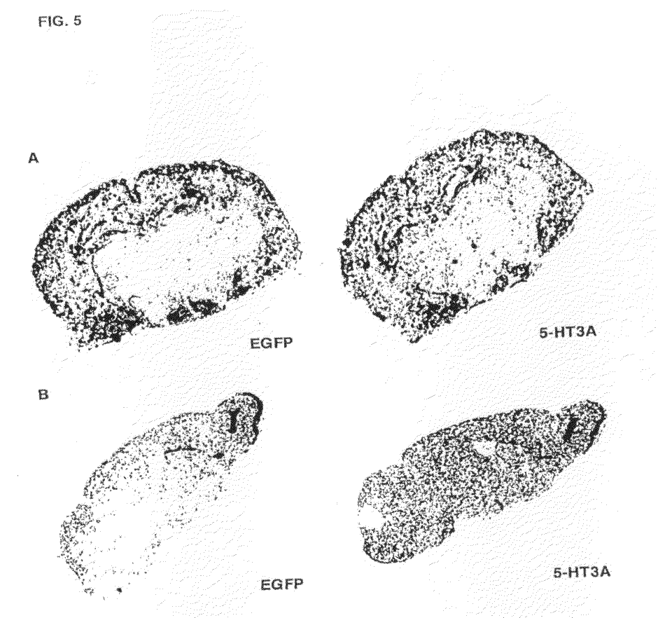

To aid the identification of 5-HT_{3A} expressing neurons, we generated a transgenic mouse model using the bacterial artificial chromosome (BAC) technology to express EGFP under the control of the 5-HT_{3A} promoter (FIG. 1A). Three transgenic lines with a similar EGFP expression 3 pattern were generated, the line with multiple copy integration of the modified BAC being used for further analysis. The specificity of EGFP expression in adult transgenic mice was indicated by in situ hybridisation (FIG. 5) and was further corroborated by electrophysiological and pharmacological methods (FIGS. 1, B and C). EGFP expression can be detected in several brain regions (mainly in hippocampus, basolateral amygdala (BLA), neocortex, OB and to a lower extent in striatum), is absent in other regions (e.g. cerebellum, thalamus) and corresponds with reported sites of 5-HT_{3A} expression (FIGS. 1, D and E).

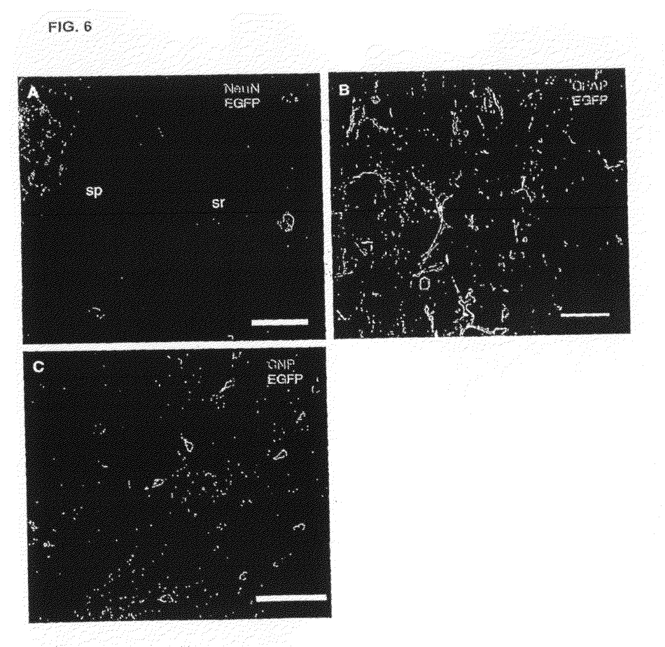

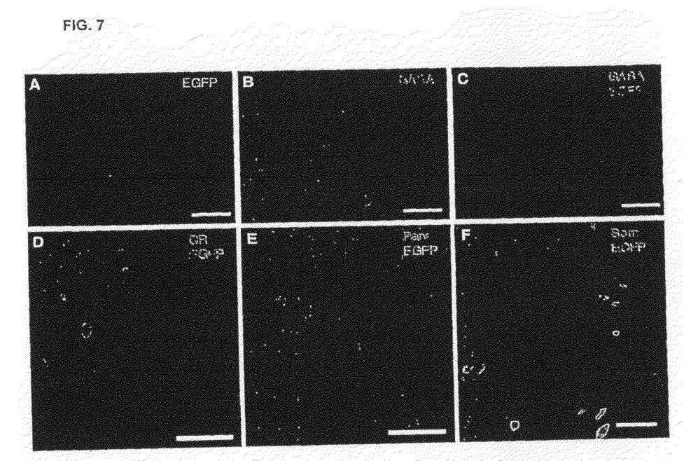

EGFP labeling occurs in NeuN-positive neurons with a distinct GABAergic phenotype and do not colocalize with the glial markers GFAP and CNP (FIG. 6). Double-labeling demonstrate that 98.2% of EGFP-positive cells in the hippocampus (n=166) and 96.9% in the cortex (n=160) co-localize with GABA (FIG. 7). There is high co-localization of EGFP expression with interneuron-specific markers such as CCK and CR but not with parvalbumin and somatostatin in young and adult mice (FIG. 7, table 1), a result that is also in agreement with previously published data.

TABLE 1

| | EGFP positive cells from all | | | | | |
|---|---|---|---|---|---|---|
| | CB | | | CB positive | CR positive | CCK |
| | positive | CR positive | CCK positive | cells | cells | positive cells |
| Region | cells | cells | cells | from all EGFP positive cells | | |
| CA1, CA3 | 77.5% | 76.9% | 60.4% | 4.2% | 15.5% | 66% |
| | (n = 89) | (n = 134) | (n = 106) | (n = 167) | (n = 187) | (n = 94) |
| Dentate | | 8.6% | | | 12.33% | |
| gyrus | | (n = 35) | | | (n = 146) | |
| Entorhinal cortex | 9.5% | 76.8% | 52.1% | 4.1% | 33.9% | 48.5% |
| | (n = 74) | (n = 99) | (n = 71) | (n = 49) | (n = 189) | (n = 66) |
| Temporal cortex | 3.1% | 65.8% | 55.6% | 1% | 31.3% | 32% |
| | (n = 65) | (n = 38) | (n = 54) | (n = 96) | (n = 112) | (n = 97) |
| Auditory | 7% | 58.5% | 43.6% | 7.7% | 23.7% | 35.6% |
| cortex | (n = 81) | (n = 65) | (n = 62) | (n = 39) | (n = 93) | (n = 59) |
| Somatosensory | 1.6% | 40.6% | 37% | 0% | 22.5% | 27.4% |
| cortex | (n = 64) | (n = 96) | (n = 46) | (n = 21) | (n = 89) | (n = 62) |

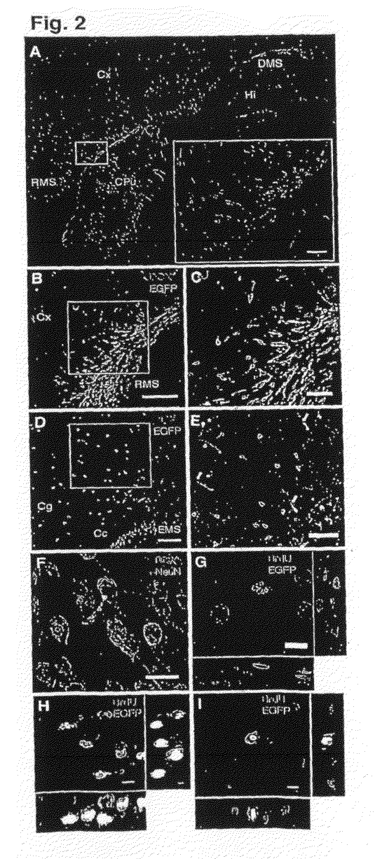

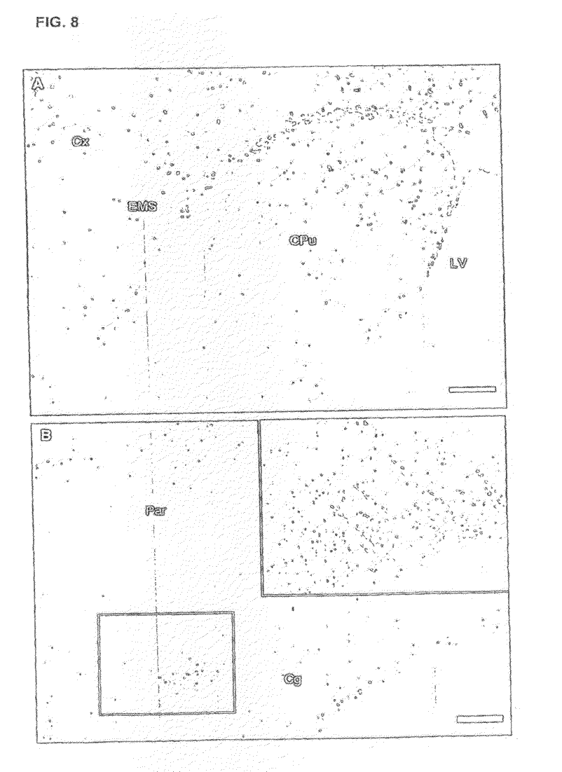

Thus, the transgenic EGFP expression recapitulates faithfully the expression of the endogenous 5-HT_{3A} gene. Unexpectedly, careful expression analysis revealed the presence of EGFP-positive cells in brain regions that are associated with postnatal neurogenesis. Thus, in situ hybridization studies indicate the presence of high mRNA levels both for 5-HT_{3A} and EGFP in the RMS in adult transgenic mice (FIG. 5). We observed in young transgenic mice, in addition to the RMS, a widespread stream of small EGFP-labeled cells that emerge from dorsal parts of the SVZ and extend along the alveus of the hippocampus (FIG. 2A). We termed this stream dorsal migratory stream (DMS). It is of note that in fixed brain slices, many EGFP-positive cells in the vicinity of the streams are oriented radially towards the cortex and appear thus to have exited the migratory streams (FIG. 2 A, inset). We detected furthermore in horizontal sections yet another stream of EGFP-positive cells that we named external migratory stream (EMS). This stream emerges from anterior parts of the SVZ and extends along the external capsule towards latero-dorsal brain regions (FIG. 8). On horizontal sections, EGFP-positive chains of cells with immature morphology can be traced all the way to superior layers of the fronto-parietal and occipital cortex (FIG. 8). Similar chains of EGFP-positive cells can also be traced from the SVZ to the nucleus accumbens and BLA. These new streams containing cells with immature morphology, indicate the presence of persistent postnatal migration that follows structures and expansions of the callosal system.

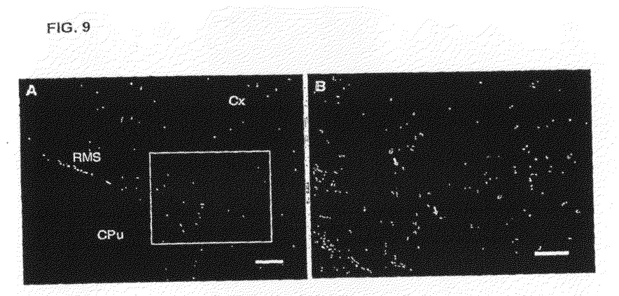

To identify the cell type of EGFP-positive cells within the streams, we performed double labelling with neuronal and glial markers. In the RMS, DMS and EMS there is no co-localization with NeuN, GFAP or CNP. We found, however, a strong co-localization of EGFP with the markers for immature neurons doublecortin (DCX) (FIG. 2, B to D), Tuj1 and PSA-NCAM. EGFP/DCX-positive cells could not only be detected in the three main streams, but also in cells that appear to have exited the streams (FIGS. 2, B, C, D and E). In the cortex EGFP/DCX-positive cells could be detected all the way up to cortical layer II. These cells exhibited various morphologies, ranging from immature cells with a leading process typical for migrating neuroblasts, to cells with a more elaborate arborization. In some of the latter, the transition from migratory into differentiated neuron was apparent since co-expression of DCX and NeuN was found (FIG. 2, F). EGFP-positive cells with the morphology of migrating neuroblasts, many co-expressing DCX, can be still detected in a significant number in the corpus callosum and in deep cortical layers also in the adult brain (FIG. 9). These cells are likely to be identical to the previously described DCX-positive cells that were found outside of the RMS/OB system. The number of EGFP/DCX-positive cells within and nearby the streams 4 clearly decreases during postnatal maturation of the brain but does not subside completely in the adult. The presence of many EGFP/DCX-positive cells within the RMS, DMS and EMS prompts the question as to their origin and time of birth. To address this question, classical birthdating studies using BrdU, a marker for DNA replication, have been performed. BrdU was injected at different embryonic stages (E12.5, E14.5, E16.5, E19.5) and on postnatal day 7 (P7) and analysis was performed one month later. In mice injected prenatally many EGFP-positive cells in the cortex (up to 60%) showed BrdU incorporation (FIG. 2G and table 2).

TABLE 2
BrdU/EGFP-double positive cells from all EGFP-positive cells

| | E 12.5 | E 14.5 | E 16.5 | E 19.5 |
|---|---|---|---|---|
| Upper cortical layers | 70% | 72.8% | 11.1% | 4.5% |
| Deep cortical layers | 46% | 47.2% | 26.1% | 20% |
| Total | 60.6% | 60% | 18.1% | 14.3% |
| | (n = 546) | (n = 590) | (n = 772) | (n = 362) |

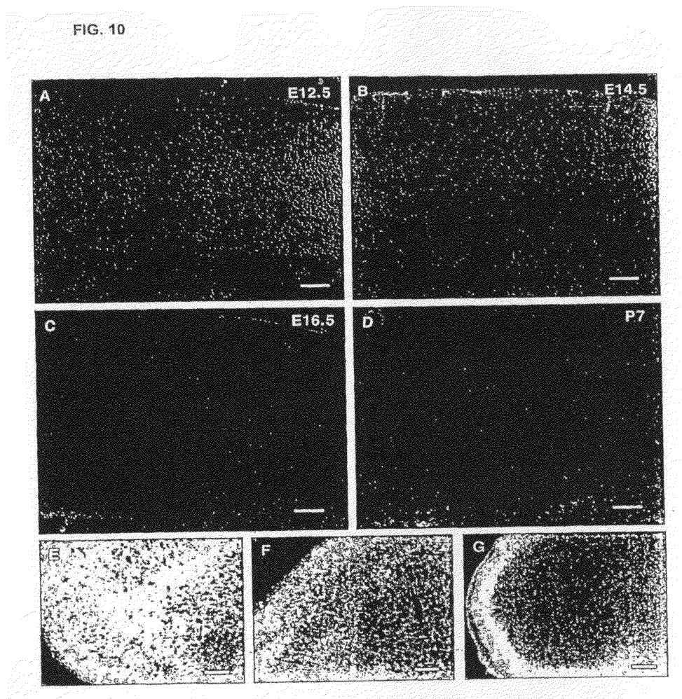

Many of the EGFP-positive cells are CR-positive and thus these data are in agreement with previous findings demonstrating that the majority of CR-positive interneurons are born in mice around E14.5. Injection of BrdU at P7 also gives rise to labeled cells within the streams and four weeks later few cells can be detected in the cortex (FIG. 10). Postnatal generation of some of the EGFP labeled cells is also indicated by the expression of the nuclear antigen Ki67, a marker for active cell proliferation, in EGFP-positive cells in the streams and in EGFP-positive cells that appear to have exited the streams (experiments were performed at P10). However, a quantitative evaluation of the prenatally versus the postnatally born EGFP-positive cell pool cannot be established since the BrdU treatment is toxic (see FIG. 10).

The origin of cells in the SVZ is still an issue of debate. Based on a large body of previous work pertaining to birth and migration of GABArgic interneurons, two scenarios can be envisaged. Since the SVZ has been shown to retain neurogenic activity in the postnatal brain, it is conceivable that EGFP/DCX-positive cells are generated postnatally within the SVZ. In primates, the SVZ provides a distinct pool of proliferative GABAergic progenitors. Alternatively, EGFP/DCX-positive cells are postmitotic and have migrated already during embryogenesis from the ganglionic eminences into the SVZ.

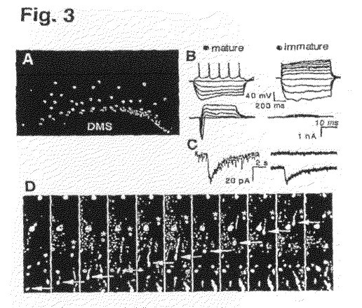

Considering that in these mice EGFP expression is controlled by the 5-HT₃ promoter, the question arose whether migrating neuroblasts express functional 5-HT₃ receptors. We thus performed patch-clamp recordings from cells with the typical migratory morphology in the DMS using acute sagittal slices from transgenic animals (FIG. 3, A). 5-HT₃ receptor-mediated inward currents following 5-HT application were readily induced in cells within the DMS. In the DMS, a ventro-dorsal ‘maturational axis’ was found, with immature neuroblasts located ventrally (adjacent to the alveus), and more mature neuroblasts located dorsally (close to the border to the white matter) (FIGS. 2, B and C). 5-HT₃ receptor-mediated inward currents following 5-HT application were also readily induced in neuroblasts that have exited the RMS. Importantly, the fact that the more ‘mature’ neuroblasts fire action potentials is additional and decisive proof for their neuronal and not glial, phenotype.

To directly demonstrate that EGFP-labeled neuroblasts exit the streams and continue to migrate into the cortex, time-lapse imaging experiments on acute slices were performed in young transgenic mice (FIG. 3E). EGFP-positive neuroblasts and their migration were followed for several hours. Movement within the streams and out of the streams can be detected already after a few minutes. EGFP-labeled cells in the streams move bi-directionally. Labeled cells in the cortical layers exhibit complex movement: the direction of many moving cells is clearly towards the pial surface but tangentional and even ventricle-directed movement is also seen. During experiments that lasted several hours neuroblasts that have exited the stream could be followed all the way up to layer IV even in slices of three week-old animals.

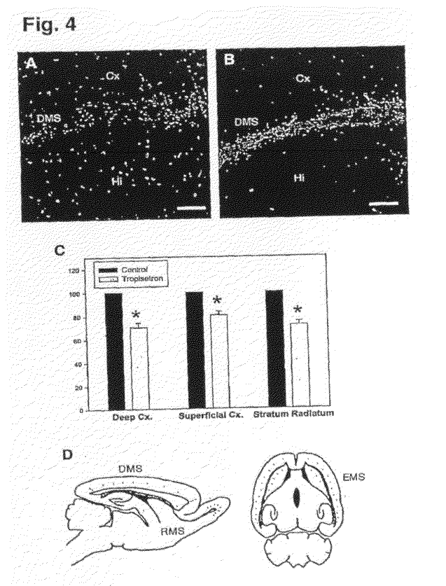

The presence of functional 5-HT₃ receptors on neuroblasts raised the question whether the receptor activation plays a role in the migration and maturation of GABAergic inhibitory neurons in postnatal life. To this end, studies were carried out in organotypic slices obtained from P3 transgenic mice (n=8) and cultured in vitro for 6 days. There were clear alterations in the cortical distribution of EGFP-positive cells in slices treated with the specific 5-HT₃ antagonist tropisetron (1 μM) compared to corresponding untreated slices (FIGS. 4, A and B). The decrease in the number of EGFP-expressing cells in tropisetron-treated animals in cortex and hippocampus indicates a deficit in the migration of these cells, rather than cell death since the neuroblasts are still clearly visible in the stream. The tropisetron-induced reduction in the number of EGFP positive cells is more accentuated in deep cortical layers (of 68.4% of the control level), but was also significant in the superficial layers of the brain (76.4%) and in the stratum radiatum of the hippocampus (72.6%). By P3 when slices were made, many EGFP-expressing cells have already reached their destination. Hence, the effect of prolonged 5-HT₃ receptor blockade on the migration of EGFP-positive cells is most likely to be even more accentuated than shown here. Similar results were obtained with lower concentrations of tropisetron (100 nM) (reduction to 70.8% of control levels in the deep cortex, 81.1% in superficial cortical layers and 74.7% in hippocampus) (n=3).

In conclusion, we describe the generation and characterization of a transgenic mouse model that has led to several novel findings. EGFP-labeled immature cells from the SVZ continue to migrate postnatally into different brain regions, including cortex, hippocampus, striatum, amygdala. Birth-dating experiments provide evidence that this cell population comprises both a postmitotic and a mitotic cell pool. The expression of neuronal markers and the presence of specific neuronal electrical properties in labeled migratory cells demonstrate that they have a neuronal fate and develop a GABAergic phenotype of a specific subtype. Neuroblasts within and in the vicinity of the migratory streams express functional 5-HT₃ receptors and their activation is important for their migration. Thus, our results demonstrate a new form of postnatal structural plasticity that is mediated by a subclass of GABAergic interneurons and modulated by 5-HT in large cortical areas.

Bipolar and double-bouquet 5-HT₃/CR-positive interneurons in layers II-III of the cortex, with long dendrites that reach all cortical layers, are major inhibitory, integrative components of cortical columns. Indeed, the streams described here may allow the proper pancortical distribution of 5-HT₃/CR-positive interneurons to cortical columns during early postnatal brain development. Ion channels, structurally related to 5-HT₃ receptors, such as GABAA and glutamate NMDA receptors, are important for migration of neuroblasts.

The findings presented herein are relevant with regard to the pathogenesis of several neuropsychiatric disorders with a neurodevelopmental background. Thus, in autism, disturbances in both the 5-HT level and GABAergic inhibition within cortical columns have been shown. Neonatal 5-HT depletion affects specifically the morphology of CR-positive interneurons. Therefore, local deficiencies of 5-HT during childhood as seen in autism, and hence impaired 5-HT₃ receptor signaling may account for altered inhibition within the cortical column microcircuits. Similar mechanisms may be responsible for reversible disturbances in regions adjacent to SVZ/RMS, rich in CR-positive interneurons that are seen in adults with major depressive and obsessive-compulsive disorders.

Elucidation of the mechanisms by which 5-HT exerts its therapeutic effects in mood disorders, acting on specific 5-HT receptors in defined neuronal populations, can advance the development of drugs that target more precisely structures of which the function is disturbed in these diseases.

### Methods

Generation of 5-HT₃-EGFP transgenic mice. Transgenic mice were generated by inserting the EGFP coding sequence into the start codon of the 5-HT_{3A} gene, following standard procedures of the BAC technology.

A. Screening of a mouse BAC library and selection of a suitable BAC, containing the 5-HT_{3A} gene. A mouse 129SV strain BAC library (Research Genetics) was screened using a 285 by probe derived from exon 9 of the 5-HT3A gene. This probe was generated by PCR using the primers 5-HT3 probe 1 (TGCAGCCATGTTGGAC) and 5-HT3 probe 2 (TCAAGAATAATGCCAAATGGAC). The size of the 5′-flanking DNA region was determined through Southern blot of a Pad-digested BAC DNA and pulse field gel electrophoresis (PFGE) analysis (CHEF-DRIII; Bio-Rad) using a 1063 by probe, contained within exon 1 and in intron 1. This probe was generated by PCR with the primers 5-HT₃-3′RA1 (CATCCCGCAGGTGCTGTTG) and 5-HT₃-3′RA2 (GCCCAGGAGGATAAATAGCA). The downstream region of the BAC DNA was determined using similar methods. The ApaI digested BAC DNA was probed with a 739 by fragment located in exon 9 and the 3′ untranslated region (3′ UTR). This was produced by PCR with the primers 5-HT3-C-3′RA (TGGCAGGGAGGGGCTATGGA) and 5-HT₃-C-3′RA2 (CGACTCGAGCCTTTCAACAT). A 10 NotI digest of the BAC DNA was performed to determine the size of the genomic insert of the BAC clones, followed by Southern blot and PFGE analysis. For PFGE, the following conditions were used: 1% agarose, 0.5× TBE, 1-6 sec switch, 6 V/cm, 120° angle, 14° C. This digest also confirmed that none of the analyzed BAC clones contained an internal NotI restriction site. From eight positive BAC clones, clone 310N1 was chosen, because it contained the largest genomic insert (approx. 195 kb with >50 kb upstream and >30 kb downstream) of the 5-HT_{3A} gene.

B. Cloning strategy for the 5′-5-HT_{3A}-EGFP construct. The EGFP coding sequence was introduced in the pIRES-EGFP vector (BD Biosciences), followed by the bovine growth hormone polyadenylation signal. The EGFP sequence was flanked by two recombination arms (RA) located around the translational start of the 5-HT_{3A} gene. These arms were necessary for mediating homologous recombination with the genomic insert in the BAC (FIG. 1). The recombination arms 5′RA and 3′RA of 1134 by and 1063 by respectively, were generated with the primers 5-HT₃-5′RA1 (TTGACCTCGGAGGGCTTGGTGC) and 5-HT₃-5′RA2 (GAGTTCCATGGCAAGCTTCCC) and the primers 5-HT₃-3′RA1 and 5-HT₃-3′RA2 (described earlier), respectively. The 5′RA was inserted in the pIRES-EGFP digested with NcoI and Bsp68I, generating pIRES-5′RA-EGFP. The 5′RA-EGFP-pA cassette was cut out from this vector with XhoI and cloned in the SmaI site of pBluescript II SK (Stratagene), generating pBS-5′RA-EGFP. The 3′recombinogenic arm was cloned in the EcoRV site of pBS-5′RA-EGFP, to create the pBS-5-HT₃-5′3′RA-EGFP final construct. The 5-HT3-5′3′RA-EGFP targeting cassette was cut out after SalI digest in this vector and inserted into the SalI site of the pSV1 recA shuttle vector that was prepared by cesiumchloride gradient purification.

C. Insertion of the 5′-5-HT_{3A}-EGFP construct into the 5-HT_{3A}-containing BAC. The homologous recombination method used to introduce the targeting cassette in the BAC clone 310N1 was previously described. For the generation of cointegrates, competent DH10B BAC competent cells were transformed with maxiprepped pSV1.RecA-5′3′RA-EGFP DNA. The cointegration and resolution steps were done as described previously. Finally, the modified BAC DNA was separated from the bacterial genomic DNA through a cesium chloride gradient ultracentrifugation. An overnight NotI digest of the resolved BAC DNA was done to release the BAC insert from the pBeloBAC11 vector backbone. The restriction digest was then loaded on a sepharose column, to isolate the released BAC insert. 0.5 ml fractions were collected from the sepharose column and subsequently checked on a PFGE gel for the integrity of the linearized BAC.

D. Production of transgenic mice. The purified and linearized BAC DNA was microinjected at a concentration of 0.7 μg/ml in B6D2F2 mouse zygotes. Founder animals were analysed to test the integrity of the integrated BAC by PCR and Southern blot. To test for the presence of the EGFP gene, EGFP-1 (ATGGTGAGCAAGGGCGAGCT) and EGFP-2 (GCCGAGAGTGATCCCGGCGGCGGT) were used. To check for the presence of both the right and left arms of the BAC, two specific primers were designed covering specific DNA sequences of the pBeloBAC11 backbone, situated between the NotI and SalI restriction sites on both sides of the genomic insert: BACL-1 (TAACTATGCGGCATCAGAGC) and BACL-2 (GCCTGCAGGTCGACTCTAGAG), BACR-1 (GTGTCACCTAAATAGCTTGGCG) and BACR-2 (GGGGTTCGCGTTGGCCGATTC). Copy number of the integrated transgene was determined by Southern blot after HindIII digestion 11 of genomic DNA and hybridisation with a 5′recombinogenic arm probe. Matings between transgenic animals and C57Bl/6 mice produced wild-type and transgenic mice in normal Mendelian ratios. The EGFP expression pattern was analysed using transgenic animals starting from the F2 generation. Electrophysiological recordings were done from transgenic animals starting from the F4 generation.

Immunohistochemistry. Immunostaining was carried out on 50 μm (or 100 μm) free-floating sections or on 300 μm organotypic slices. The following primary monoclonal antibodies were used in combination with the polyclonal rabbit anti-EGFP antibody, 1:10000 (Molecular Probes) or the monoclonal anti-EGFP antibody, 1:300 (Chemicon): goat anti-doublecortin antibody, 1:500 (SantaCruz), rabbit anti-GABA antibodies, 1:1000 (Sigma), mouse anti-cholecystokinin antibody, 1:5000 (UCLA RIA Core), mouse anti-parvalbumin antibody, 1:3000 (Sigma), rat antisomatostatin antibody, 1:1000 (Chemicon), mouse anti-calretinin antibody, 1:5000 (Swant), mouse anti-calbindin antibody, 1:5000 (Swant), mouse anti- glial fibrillary acidic protein (GFAP), 1:100 (Chemicon), mouse anti-NeuN, 1:1000 (Chemicon), mouse anti-PSA-NCAM, 1:500 (Chemicon), mouse anti-III class α-tubulin, Tuj1, 1:500 (Covance), anti-Ki67, 1:500 (Abcam), and mouse anti-2′,3′-cyclic nucleotide 3′-phosphodiesterase (CNP), 1:5000 (Stemberger).

For visualization of primary antibodies, slices were incubated with FITC- or Alexa 488-conjugated (Molecular Probes) conjugated anti-rabbit IgG and anti-mouse, anti-goat or antirat Cy3 coupled secondary antibody (Jackson Immuno Research Laboratories). For BrdU-EGFP double labelling, sections were incubated sequentially with polyclonal anti-EGFP antibody overnight, incubated with a biotinylated secondary antibody, 1:500 (Vector Laboratory), re-fixed in 4% PFA and treated with 2N HCl for 30 min at 37° C. to denature the DNA. Further, slices were incubated with anti-BrdU antibody, 1:500 (Accurate) overnight and treated with Cy3-conjugated secondary antibody and streptavidin-FITC, 1:500 (Vector Laboratory). Sections were analyzed using an upright fluorescent microscope (Zeiss Axioplan 2) or an LEICA TCS-NT confocal microscope. The antibody #9303 raised against Gastrin/CCK was provided by Antibody/RIA Core, CURE/Digestive Diseases Center, Los Angeles, Calif.

Birthdating analysis. Pregnant mice and P7 transgenic mice were injected intraperitoneally with 50 mg/kg or 300 mg/kg BrdU (Sigma) (body weight), sacrificed after 1 or 4 weeks and processed for immunostaining.

BrdU was shown to persist in the circulation less than a few hours after intraperitoneal injection. Hence a single BrdU injection provides only a snapshot of proliferating cells. Five BrdU injections (50 mg/kg, intraperitoneally) were administered every 2 hrs apart for 8 hours at E12.5, E14.5, E16.5 and P7 (FIG. 10). Differences in blood flow, metabolism and the development of the blood-brain-barrier were mentioned as factors that reduce the access of BrdU into the brain when injected postnatally. To avoid a cumulative effect, 5 pulses of BrdU (one pulse per day) were administered from P7 to P12. At P7 two treatments, one with 50 mg/kg and with 300 mg/kg of BrdU were carried. The latter was chosen because this dose was suggested to effectively label newborn neurons in the adult brain. Following all treatments, toxic effects were clearly noticeable. Hair loss and motor deficits are apparent in P16 mice. The toxic effect was dose-dependent: a more pronounced phenotype is visible at higher doses (300 mg/kg) than at lower doses (50 mg/kg). Most importantly, we observed a reduction in the number of EGFP-positive cells in the cortex, the RMS and OB at P16 after postnatal treatment with BrdU (FIG. 10). In the OB the dose-dependent neurotoxic effect on newborn neurons can be seen already by light microscopic inspection. Injections with BrdU were shown to have general toxic and specifically neurotoxic effects in young animals: Even at low levels BrdU was reported to inhibit neurogenesis. Thus, the toxicity of BrdU when used at postnatal stages precludes a quantitative evaluation of birthdating experiments.

In situ hybridization. Radioactive in situ hybridization experiments were performed essentially as described (2), using [γ-³⁵S] dATP-end-labeled oligonucleotides corresponded to nucleotides 181-225 and 334-378 of the mouse 5-HT_{3A} and EGFP coding sequence, respectively.

Slice preparation and electrophysiology. 250-350 μm-thick parasagittal brain sections (for recordings in hippocampus, cortex, RMS and DMS) or coronal brain sections (for recordings in amygdala) were made from transgenic mice (P8-P42) using a vibratome (Leica VT1000S). Whole-cell voltage and current clamp recordings were made from visually identified, EGFPpositive neurons using and EPC9 amplifier (HEKA) and an upright microscope (Zeiss Axioskop FS2) equipped with IR-DIC and standard epifluorescence. Patch pipettes had a resistance of 2-6 MΩ when filled with (in mM): 105 K-gluconate, 30 KCl, 0.5 CaCl₂, 5 EGTA, 2 Mg-ATP, 10 HEPES. Slices were continuously superfused with ACSF containing (in mM): 120 NaCl, 3.5 KCl, 2.5 CaCl₂, 1.3 MgSO₄, 1.25 NaH₂PO₄, 25 NaHCO₃, 25 glucose. The presence of functional 5-HT3 receptors was assessed by application of 100 μM 5-HT (dissolved in ACSF) via pressure 12 ejection from a second pipette connected to a picospritzer. Cells were voltage clamped at −60 mV. Signals were filtered at 1-5 kHz, sampled at 2-10 kHz and off-line analysis was performed using Igor Pro (Wavemetrics).

Time lapse imaging. Sagittal slices were generated as described for electrophysiological experiments at 250 μM thickness. A stack of 21 frames (spanning 100 μM) was taken every minute with an Olympus microscope and a 20X/0.5NA objective. A focus projection of the stack was generated using ImageJ for visualization of cells throughout the stack.

Organotypic slice culture experiments. Organotypic slice cultures were performed as previously described. After 6 days in culture, slices, control or treated with tropisetron (Sigma), were fixed for 1 hr in 4% PFA and processed for immunostaining using polyclonal rabbit anti-EGFP antibody. Cell counts were performed in pairs of matching tropisetron-treated and untreated slices. EGFP-positive cells were counted in deep, in superficial cortical layers and in stratum radiatum of the hippocampus within three standardized rectangular regions of 200×150 μm₂ for each region. Data from control and treated slices were analyzed by Student's t test.

What is claimed is:

1. A method of screening a candidate agent for modulation of post-natal neuroblast migration, the method comprising:
contacting a 5-HT_{3A} receptor with a candidate agent;
determining if said agent specifically interacts with said 5-HT_{3A} receptor;
administering said agent to an animal in a model for post-natal neuroblast migration;
determining if said agent modulates post-natal neuroblast migration.

2. A method of modulating post-natal neuroblast migration in an individual mammal, said method comprising:
administering to said individual mammal an effective amount of a modulator of 5-HT_{3A} receptor activity.

3. The method according to claim 2, wherein said modulator of 5-HT_{3A} receptor activity is an antagonist.

4. The method according to claim 2, wherein said modulator of 5-HT_{3A} receptor activity is an agonist.

5. The method according to claim 2, wherein said modulator of 5-HT_{3A} receptor activity is selectively acts on the 5-HT_{3A} receptor as compared to other serotonin receptors.

6. The method according to claim 2, wherein said individual is human.

7. The method according to claim 6, wherein said human is autistic.

8. A kit for use in modulating post-natal neuroblast migration of an individual mammal, said kit comprising:
an effective dose of a modulator of 5-HT_{3A} receptor activity, in a pharmaceutically acceptable vehicle.
